(19) United States
(12) Patent Application Publication
(10) Pub. No.: US 2009/0099656 A1
(43) Pub. Date: Apr. 16, 2009

(54) Method and device for a breast implant

(76) Inventors: Hatuna Gelda, Ashdod (IL); Uzi Ezra Havosha, Tel Aviv (IL)

Correspondence Address: UZI EZRA HAVOSHA & PARTNERS, HADAR-DAFNA HOUSE 39 SHAOUL HAMELECH STREET, TEL AVIV, 64928, IL

(21) Appl. No.: 11/974,416

(22) Filed: Oct. 15, 2007

(51) Int. Cl. A61F 2/12

(52) U.S. Cl. 623/8

(57) ABSTRACT

A breast implant with outer spherical layers, layer of silicon gel and an inner core of gas filled silicon spheres. In the outer spherical layers are pairs of expandable pockets comprising an inner and an outer pocket with a connecting tube accessible from outside the body. The pockets are for post operation additional breast enlargements without major surgery. Option for the inner core silicon spheres to be filled with a light and flexible substance like for example, a sponge-like substance.

### FIELD OF THE INVENTION

This invention is in the field of plastic surgery and in particular surgery to implant a breast enlarger with the ability to make further enlargements without major surgery.

### BACKGROUND OF THE INVENTION

Women may wish to have a breast enlargement for a number of reasons for example, the natural breasts are small or there is fashion in women's clothes that encourages large breasts. Women who have had surgery for removal or partially removal breasts may opt to replace the missing part to be implanted with an artificial breast. Another case of requiring this kind of surgery is where a woman has sagging breasts as a result of old age or weight loss and the woman wishes to restore her former appearance.

One of the difficulties women experience when having a breast enlargement is the psychological adjustment to a significant increase in size. Surveys in the United States of America and Denmark show that women who have had breast implants are more likely to have problems of depression. It would help women to overcome this reaction to the implant if they could have the enlargement done in stages. In such a case it would be very desirable to be able to avoid surgery for the later enlargements.

U.S. Pat. No. 6,875,233 issued to Turner shows a method of post operative breast size changing. However this patent claims a filling valve that remains external to the patient which is highly undesirable. The present invention addresses this difficulty.

The company Mentor Medical Systems markets a breast expander with filling ports that remain outside the body. This solution is undesirable from the physiological and aesthetic viewpoints. The present invention takes care of this problem.

Another problem with prior art implantations is that the weight of implants causes healing problems after surgery and increases the sag effect on the breast which is the opposite of the desired result of the implant, namely to raise the breast and give it a fuller appearance. Some implants use silicon as a filler and some use a saline solution. Prior art does reveal a gas filling but not a combination of gas and solid to provide the desired weight and texture of the implanted breast.

A disadvantage of saline and gas fillings is their propensity to leak from the prosthesis into the woman's body. The present invention addresses this problem.

### SUMMARY OF THE INVENTION

It is to be understood that both the foregoing general description and the following detailed description present embodiments of the invention and are intended to provide an overview or framework for understanding the nature and character of the invention as it is claimed. The accompanying drawings are included to provide a further understanding of the invention and are incorporated into and constitute a part of this specification. The drawings illustrate various embodiments of the invention and, together with the description, serve to explain the principles and operations of the invention but not to limit the invention to these descriptions only.

The object of this invention is to implant an artificial breast or part of a breast in order to replace a removed breast or to enlarge or change the shape of an existing breast.

Another object of this invention is for the implanted breast to be lighter in weight than the natural weight of breasts.

Another object of this invention is for the texture and consistency of the implant to be close to the texture and consistency of a natural breast.

Another object of this invention is for a portion of the filling of the prosthesis to be silicon and a portion air filled spherically shaped silicon pieces. The latter air filled pieces would be approximately one third, by volume, silicon and two thirds, by volume, air or other suitable gas. An alternative to filling the spherically shaped silicon pieces is to fill the silicon pieces with a light and flexible substance for example a sponge-like substance.

Another object of this invention is for there to be in the implantation at least one empty pocket with a connecting tube to fill from outside the breast. Such a pocket would be made and attached to the structure of the prosthesis in such a way that the pocket expands primarily on the pocket's upper side when filled with a substance like silicon.

Where for example two pockets are used, there could be one pocket positioned on the lower left side of the breast and the other on the lower right side of the breast. The lower surface of the pocket could be attached to the lower portion of the shell of the implant so that the upper portion of the pocket would be free to expand. This arrangement would make the prosthesis expand in an upward direction when the pocket is filled with silicon and hence make the whole breast appear higher and fuller than previously.

Another object of this invention is to enable further enlargement of the breast after the initial implantation surgery where the further enlargement does not require regular surgery. The further enlargement may require minor surgery just to access the lead tubes that lead to the fillable pocket.

Another object of this invention is for there to be an option for the further enlargement to be done more than once.

Another object of this invention is for the substance of the implant or at least its outer layer to contain an additive that is visible by electromagnetic photography like x-ray and computed tomography photography. This will enable the examiner of such photographs to differentiate clearly between areas of the natural breast and the boundaries of the prosthesis.

### BRIEF DESCRIPTION OF THE DRAWINGS

The accompanying drawings, which are incorporated in and form a part of this specification, illustrate embodiments of the invention and together with the description, serve to explain by way of example only, the principles of the invention:

FIG. 1 is a schematic depiction of the side sectional view of a breast implant.

FIG. 2 is a schematic depiction of the post surgery expansion pockets. FIGS. 2a, 2b and 2c show the stages of filling the post surgery expansion pockets.

FIG. 3 is a schematic depiction of post implant surgery with the position of the expansion pockets in the breast showing before and after the pockets have been expanded.

FIG. 4 is a schematic depiction of the situation as in FIG. 3 from an angle showing the front and the side of the patient.

### DETAILED DESCRIPTION OF THE PREFERRED EMBODIMENTS

As will be appreciated the present invention is capable of other and different embodiments than those discussed above and described in more detail below, and its several details are capable of modifications in various aspects, all without departing from the spirit of the invention. Accordingly, the drawings and description of the embodiments set forth below are to be regarded as illustrative in nature and not restrictive.

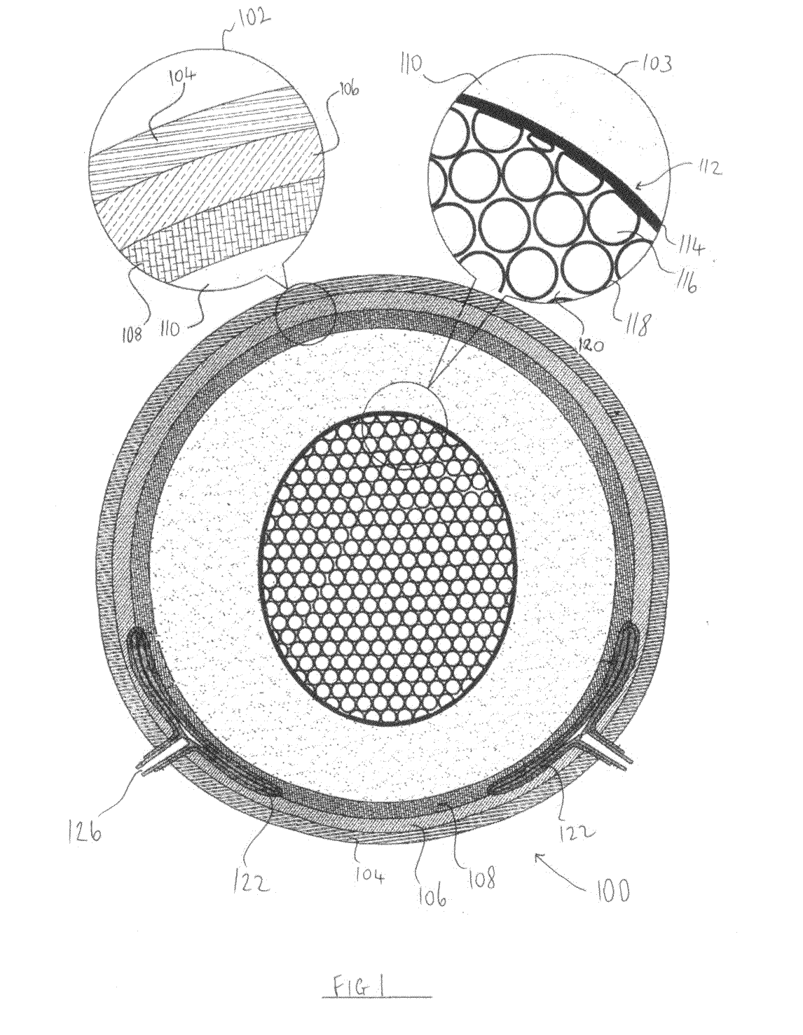

FIG. 1 shows the basic components of the breast implant 100. The outer shell is shown in the magnified diagram 102. The outer-most layer 104 and the adjacent layer 106 are made of a harder type of silicon. The next layer 108 is made of a more flexible silicon. Beyond the shell 108 is a silicon gel filler 110.

The silicon filler 110 fills the space until the core section 112 as shown in the magnified diagram 103. The core is surrounded by a harder type of silicon layer 114. Inside the core 112 are balls 116 filled with air, other suitable gas or a light and flexible substance like for example, a sponge-like substance. The external wall 118 of each ball is made of silicon that is impervious to gas. The space 120 between the balls is air or other suitable gas.

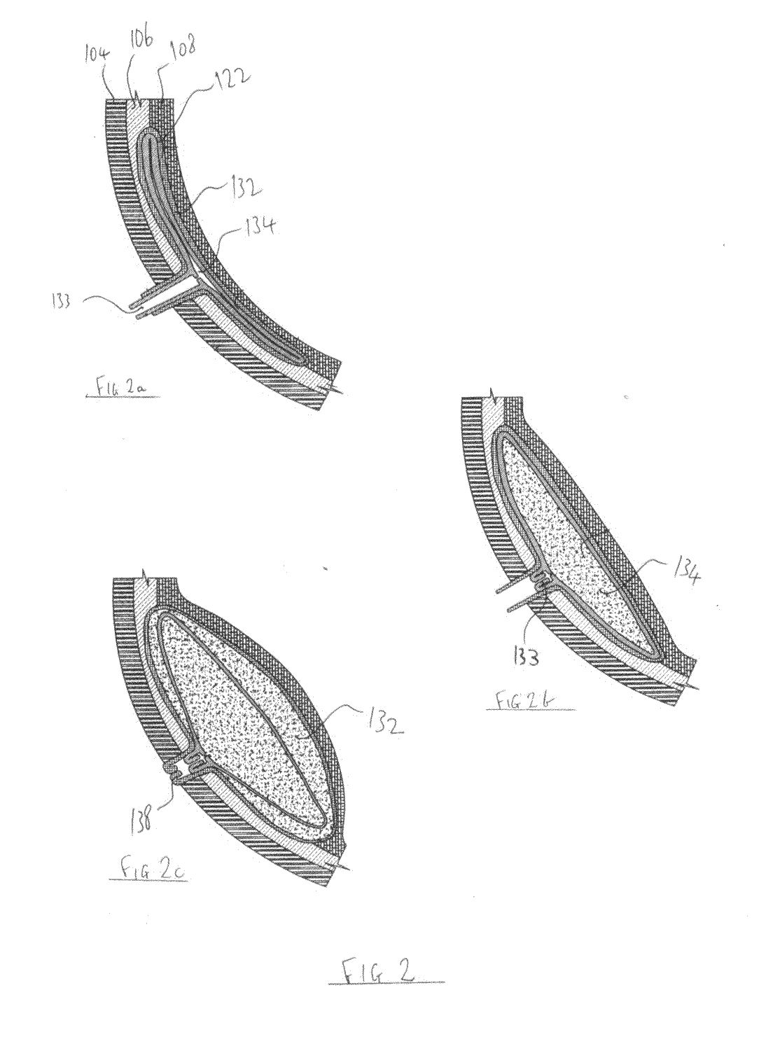

On the lower side of the implant are four expandable pockets divided into two pairs of pockets 122. Each pocket has a smaller pocket within the larger pocket. This is shown in greater detail in FIG. 2. The pair of pockets 122 lies between layers 106 and 108. The entrance valve 126 is used to insert silicon which causes the pocket 122 to expand thereby pushing the silicon 110 and the inner core 112 in an upward direction. The expansion of the pocket 122 is prevented from expanding in a downward direction by the less flexible silicon layers 104 and 106.

Two pairs of pockets are on the lower outside portion of the implant. That is to say the sectional view of FIG. 1 is not looking frontally at the person with the implant 100 but a side sectional view. One pocket is placed in the lower outer portion of the implant and the other is placed in the lower inner portion of the implant being nearer to the body of person. The final position of these pockets can be decided by the person having the implant and her medical advisors. The factors considered could be the wishes of the patient how she wants the final shape of her breasts to be, given the size and shape of her natural breasts.

FIG. 2 shows the steps involved filling the pockets 122 with silicon.

FIG. 2a shows an outer pocket 132 whose size could be around 50 milliliters and an inner pocket 134 whose size could be around 20 milliliters.

There is a valve 133 that allows a substance like silicon to be pumped in to the inner pocket 134. The valve 133 can be sealed after the silicon has been inserted. Optionally air or other gas could be used to fill the pockets. The less flexible silicon layers 104 and 106 hold the shape of the outer rim of the insert FIG. 1 100 when the pocket 122 is expanded with silicon. The silicon layer 108 is more flexible in order to allow the implant to expand upward when the pocket 122 is placed in the lower portion of the implant FIG. 1 100.

FIG. 2b shows the inner pocket 134 filled and the valve 133 sealed. The result of filling the pocket 134 is the movement of the silicon filler FIG. 1 110 inward towards the center of the implant 1 100 which in turn pushes the inner core FIG. 1 112 in the same direction. The pocket 122 is placed in the lower section of the implant so that the effect of filling the inner pocket 134 with silicon is to push the breast in an upward direction thereby giving the breast a fuller appearance.

FIG. 2c shows the outer pocket 132 filled and the outer valve 138 sealed. The effect of filling the outer pocket 132 with a filling is to push the breast further in the upward direction and inward towards the other breast.

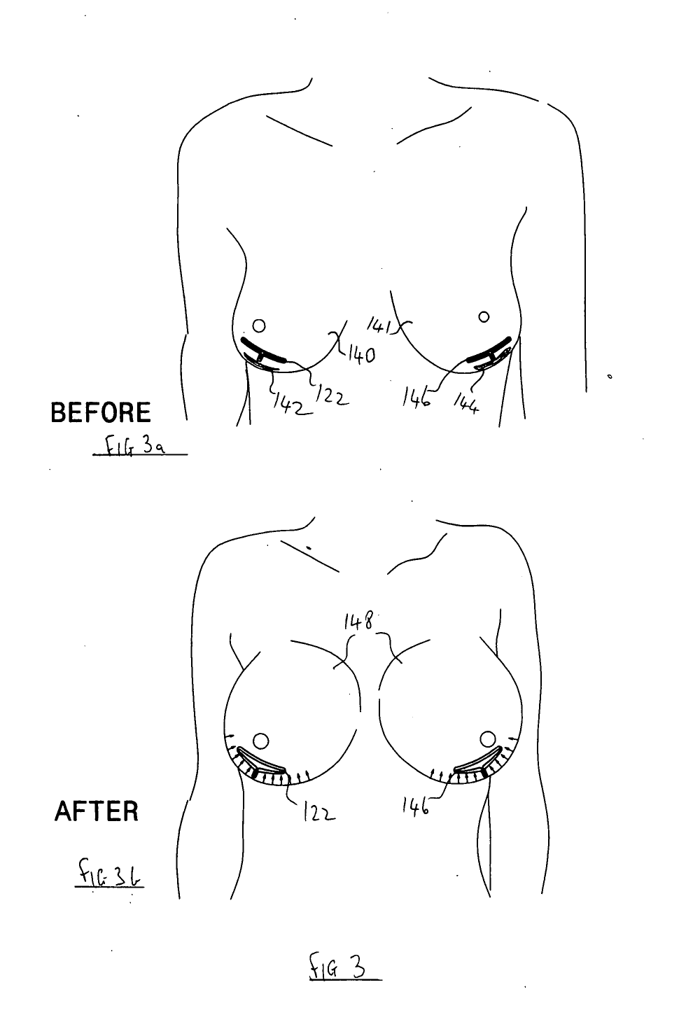

FIG. 3 shows a women after the implant operation but before the pockets 122 are expanded. This is shown in the FIG. 3a. The implant FIG. 1 100 is placed in the lower portion of the breast 140. FIG. 3b shows the expanded breasts after the expansion pockets have been filled and expanded.

FIG. 3a shows a total of eight expansion pockets. Each of the four expansion pockets shown has an inner and outer pocket as described in FIG. 2.

The implant operation has the effect of making the breasts 140 and 141 appear enlarged. When the patient wishes to further expand her breasts this would normally be done in the following order. The inner pocket FIG. 2 134 of the pocket 142 nearer to the women's body is the first to be filled. Next to be filled is the equivalent pocket 144 on the other breast. Next to be filled is the inner pocket of the pocket 122 further from the woman's body and the equivalent pocket 146 on the other breast.

At this stage all four inner pockets have been filled. Then the four outer pockets FIG. 2 132 are filled in the same order as the inner pockets were filled.

FIG. 3a shows the shape of the woman's breasts before the post operational expansions whereas FIG. 3b shows the shape of the woman's breasts after the post operational expansions. FIG. 3b only shows expansion pockets 122 and 146. The expansion pockets 142 and 144 are closer to the woman's body and not visible in FIG. 3b.

FIG. 3b shows the effect of all eight expansion pockets being filled, namely the breasts bulge 148 at their upper and inner portion.

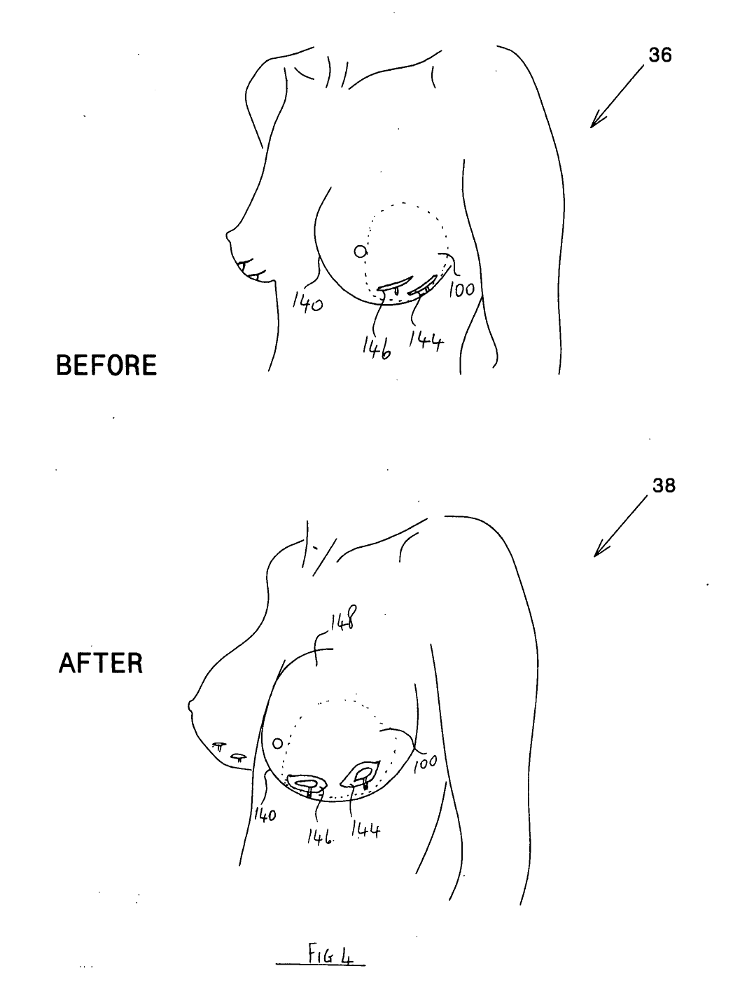

FIG. 4 shows the women patient from an angle showing the front and side view.

The depiction marked “before” is the situation after the implant 100 where the expansion pockets 144 and 146 are in their contracted form. The pocket 144 is placed on the lower side of the breast 140 nearer to the woman's body and the other pocket 146 is placed further from the woman's body, both being on the farther portion of the breast 140 from the other breast. The effect of expanding the pockets and 146 from these lower outer positions cause the breast to expand in an upper and inner direction giving the full appearance 148 as seen in the depiction marked “after”.

The present invention is not intended to be limited to the embodiments described above, but to encompass any and all embodiments within the scope of the following claims.

What is claimed:

1. A breast implant comprising,
a. two spherical layers of a firm impervious silicon compound,
b. a third spherical layer of silicon compound on the inside of the said two spherical layers,
c. a spherical layer of silicon gel on the inside of the said third spherical layer,
d. a central core in the said silicon gel surrounded by a spherical layer of silicon compound containing silicon spheres filled with a sponge-like substance, and
e. at least one expandable pocket with a sealable connecting tube for filling purposes and accessible from outside the body,
whereby enlargement implants can be inserted in female breasts with the option for future enlargement without surgery.

2. A breast implant as claimed in claim 1 wherein the said sponge-like substance is replaced by gas.

3. A breast implant as claimed in claim 1 wherein the said sponge-like substance is replaced by air.

4. A breast implant as claimed in claim 1 wherein the said pocket is a pocket within a pocket where each said pocket is independently accessible from outside the body for the purpose of filling.

5. A breast implant as claimed in claim 4 wherein the said filling is a silicon compound.

6. A breast implant as claimed in claim 1 wherein at least one layer of the said two spherical layers contains a compound that is visible in electromagnetic photograph results.

7. A breast implant as claimed in claim 1 wherein the said third spherical silicon layer is a softer silicon than the said two outer spherical layers whereby the said third spherical layer will enable the said expandable pocket to expand in the direction of the center of the said breast implant when the said pocket is filled thereby raising the level of the natural breast.

8. A breast implant as claimed in claim 1 wherein the said accessibility from outside the body involves minor surgery to break the skin to reach the said connecting tubes.

9. A breast implant as claimed in claim 1 wherein the said two spherical layers of silicon are one spherical layer of silicon.

10. A breast implant as claimed in claim 1 wherein the said spherical layers are not exactly spherical but distorted spherical type shapes.

11. A breast implant comprising,
a. at least one spherical layer of an impervious silicon compound,
b. a spherical layer of silicon gel on the inside of the said spherical layer of silicon compound,
c. a central core in the said silicon gel containing silicon spheres filled with a sponge-like substance, and
d. at least one expandable pocket with a sealable connecting tube for filling purposes and accessible from outside the body,
whereby enlargement implants can be inserted in female breasts with the option for future enlargement without surgery.

12. A breast implant as claimed in claim 11 wherein the said sponge-like substance is replaced by gas.

13. A breast implant as claimed in claim 11 wherein the said sponge-like substance is replaced by air.

14. A breast implant as claimed in claim 11 wherein the said pocket is a pocket within a pocket where each said pocket is independently accessible from outside the body for the purpose of filling.

15. A breast implant as claimed in claim 14 wherein the said filling is a silicon compound.

16. A breast implant as claimed in claim 14 wherein the said filling is a sponge-like substance.

17. A breast implant as claimed in claim 11 wherein at least one layer of the said spherical layers contains a compound that is visible in electromagnetic photograph results.

18. A breast implant as claimed in claim 11 wherein the said accessibility from outside the body involves minor surgery to break the skin to reach the said connecting tubes.

19. A breast implant as claimed in claim 11 wherein the said spherical layers are not exactly spherical but distorted spherical type shapes.
